(19) United States
(12) Patent Application Publication
(10) Pub. No.: US 2025/0154469 A1
(43) Pub. Date: May 15, 2025

(54) FREEZE-DRYING PROTECTIVE AGENT FOR MESENCHYMAL STEM CELL-EXOSOMES, AND APPLICATION THEREOF

(71) Applicants: Kanbio Medtech Company, Ltd., Guangdong (CN); AIE Bioscience (Guangdong) Co., LTD., Guangdong (CN)

(72) Inventors: Xin WEI, Guangdong (CN); Shuxia WU, Guangdong (CN); Guohua TAN, Guangdong (CN); Yuxia LI, Guangdong (CN); Jiaqing SHAO, Guangdong (CN)

(73) Assignees: Kanbio Medtech Company, Ltd., Guangdong (CN); AIE Bioscience (Guangdong) Co., LTD., Guangdong (CN)

(21) Appl. No.: 18/935,653

(22) Filed: Nov. 4, 2024

(30) Foreign Application Priority Data
Nov. 14, 2023 (CN) 202311513233.7

(51) Int. Cl. C12N 5/0775; A61K 35/28

(52) U.S. Cl. CPC C12N 5/0662; A61K 35/28

(57) ABSTRACT

The present disclosure belongs to the field of exosome lyophilization protection technology and discloses a freeze-drying protective agent for mesenchymal stem cell-exosomes and application thereof, which is composed of 0.1-0.2 v/v % ceramide, lysine, and 0.8-1.5 v/v % glycerol. After mixing the freeze-drying protective agent with the exosomes, a final concentration of the lysine is 50-80 mM. The freeze-drying protective agent can maintain the physical integrity of exosome, avoid exosome aggregation, and maintain the biological function of exosome, which can be used in exosome freeze-drying technology with wide application value.

### CROSS-REFERENCE TO RELATED APPLICATION

This application claims the priority benefit of China application serial no. 202311513233.7, filed on Nov. 14, 2023. The entirety of the above-mentioned patent application is hereby incorporated by reference herein and made a part of this specification.

### BACKGROUND

### Technical Field

The present invention belongs to the field of lyophilization protection of exosomes, and more specifically, to a freeze-drying protective agent for mesenchymal stem cell-exosomes (MSC-exosomes), and application thereof.

### Description of Related Art

Exosomes are small membrane vesicles (30-150 nm) containing complex RNAs and proteins, which specifically refer to disciform vesicles with diameters of 40-100 nm. Exosomes were first identified in sheep reticulocytes in 1983 and were named “exosomes” by Johnstone in 1987. Various cells can secrete exosomes in both normal and pathological states. It is mainly derived from poly-vesicle bodies formed by the invagination of intracellular lysosomal microparticles, which are released into the extracellular matrix after fusion of poly-vesicle outer membrane and intracellular membrane. Exosomes are secreted from all cultured cell types, and exosomes are naturally found in body fluids, including blood, saliva, urine, cerebrospinal fluid, and milk. Nowadays, exosomes are found to have diverse functions because of a large number of studies on their biological sources, their material composition and transport, intercellular signal conduction, and their distribution in body fluids. The function of exosomes depends on the cell type from which they originate, which can participate in the immune response, antigen presentation, cell migration, cell differentiation, tumor invasion, and other aspects.

The storage of exosomes is of great significance, if there is no qualified storage technology, they cannot achieve long-term storage. The existing research found that −80° C. is the best for long-term storage temperature. The loss of biologically active proteins (including exosomes) can be effectively inhibited at this temperature, but under such storage conditions, a certain degree of change may still take place in the properties of exosome, and it is difficult to achieve stable transportation and storage of exosomes at this temperature. Lyophilization is usually used to preserve biologically active compounds and has now been used in the storage of exosomes. The existing reported freeze-drying protective agents for exosomes include polysaccharides such as trehalose and mannitol, alcohols, and other substances, but it has been found in a variety of studies that the freeze-drying protective agents used for specific types of exosomes are different, and the known freeze-drying protective agent can not better protect the physical integrity of the exosome, which greatly affect the biological function of the exosome.

Exosomes are derived from mesenchymal stem cells, which are rich in more than 100 extracellular matrix proteins, mRNA messenger ribonucleic acids, and multiple growth factors. Clinically, stem cell exosomes are mainly used to treat burns, scalds, skin ulcer, and to regenerate healthy skin. In the skin care aspect, they are mainly used for repairing skin damaged by trauma, aging, and poisoning, comprehensively regulating the skin, improving skin quality, and restoring the skin to a youthful and healthy state. MSCs exosomes are a current topic in the field of dermatology. However, existing technologies currently lack freeze-drying protective agent targeted at MSC-exosomes.

### SUMMARY

To overcome the above problems existing in the prior art, the present invention first provides a freeze-drying protective agent for mesenchymal stem cell-exosomes (MSC-exosomes).

A second object of the present invention is to provide application of the above-described freeze-drying protective agent.

A third object of the invention is to provide a mesenchymal stem cell-exosome freeze-drying preparation.

The object of the present invention is realized by the following:

A freeze-drying protective agent for mesenchymal stem cell-exosomes consists of 0.1 to 0.2 v/v % ceramide, lysine, and 0.8 to 0.1.5 v/v % glycerol, wherein a final concentration of the lysine after mixing the freeze-drying protective agent with the mesenchymal stem cell-exosomes is 50 to 80 mM.

Preferably, the freeze-drying protective agent for mesenchymal stem cell-exosomes is composed of 0.18 v/v % ceramide, lysine, and 1.3 v/v % glycerol, wherein the final concentration of the lysine after mixing the freeze-drying protective agent with the mesenchymal stem cell-exosomes is 70 mM.

The invention also provides application of the above freeze-drying protective agent in preparing an exosome freeze-drying preparation.

The invention also provides application of the above freeze-drying protective agent in improvement on exosome morphology.

Preferably, the improvement on exosome morphology refers to increasing a dispersion degree of exosome and/or decreasing exosome membrane fusion.

The invention also provides a mesenchymal stem cell-exosome freeze-drying preparation containing the above freeze-drying protective agent for mesenchymal stem cell-exosomes.

Compared with the prior art, the present invention has the following beneficial effects:

The invention provides a freeze-drying protectant (a protective agent) for mesenchymal stem cell-exosomes, composed of 0.1-0.2 v/v % ceramide, lysine, and 0.8-1.5 v/v % glycerol. After mixing the exosome protective agent with the exosomes, the final concentration of lysine is 50-80 mM. The exosome protective agent maintains physical integrity, avoids exosome aggregation, and can maintain the biological function of exosome, which can be used in exosome freeze-drying technology, and has wide application value.

### BRIEF DESCRIPTION OF THE DRAWINGS

FIG. 1A and FIG. 1B show exosome EM photos; the ruler in FIG. 1A is 200 nm and the ruler in FIG. 1B is 600 nm;

FIG. 2 shows an exosome particle size analysis map;

FIG. 3 shows an experimental picture of wound repair by the exosome.

### DESCRIPTION OF THE EMBODIMENTS

The specific examples of the present invention will be further explained below. It should be noted here that the description of these examples is used to aid in the understanding of the invention, but it does not constitute a limitation to the invention. Furthermore, the technical features described below in the respective examples of the present invention can be combined as long as there is no conflict with each other.

1. Preparation of Exosomes from Mesenchymal Stem Cells. The Operation is as Follows:

(1) Collecting a first supernatant of 108 MSC s grown to 80% fusion degree, centrifuging at 3000 g, and collecting a second supernatant;

(2) Subjecting the second supernatant in (1) to centrifugation at 3000 g to remove cell debris, and collecting a third supernatant;

(3) Subjecting the third supernatant in (2) to centrifugation at 10000 g to remove proteins, collecting a fourth supernatant, and filtering the fourth supernatant with 0.22 μm filter membrane to obtain a fifth supernatant;

(4) Subjecting the fifth supernatant to ultracentrifugation at 100000 g for 1.5 h, discarding an obtained supernatant, resuspending an obtained precipitation with PBS, and collecting a resuspension; and

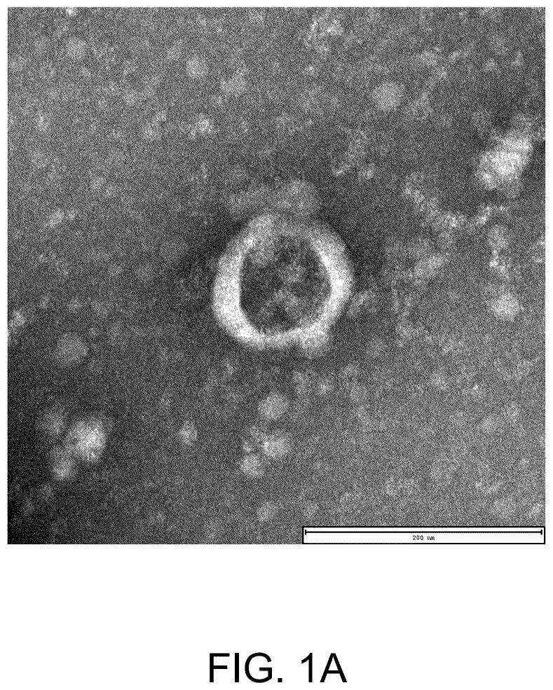

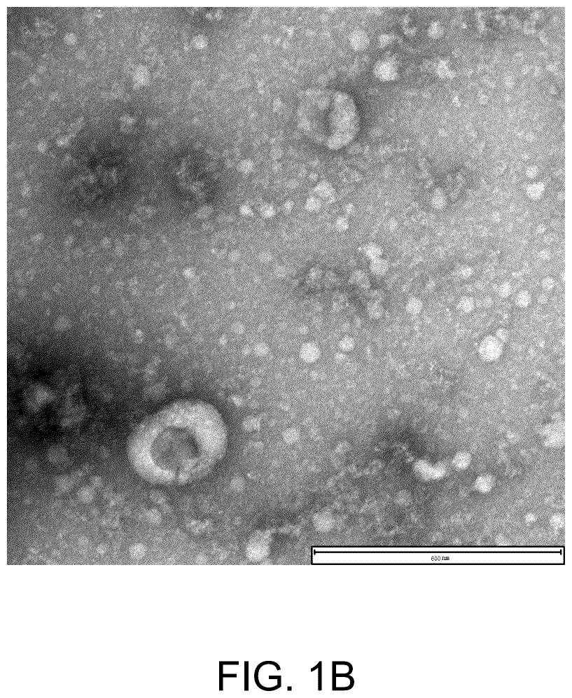

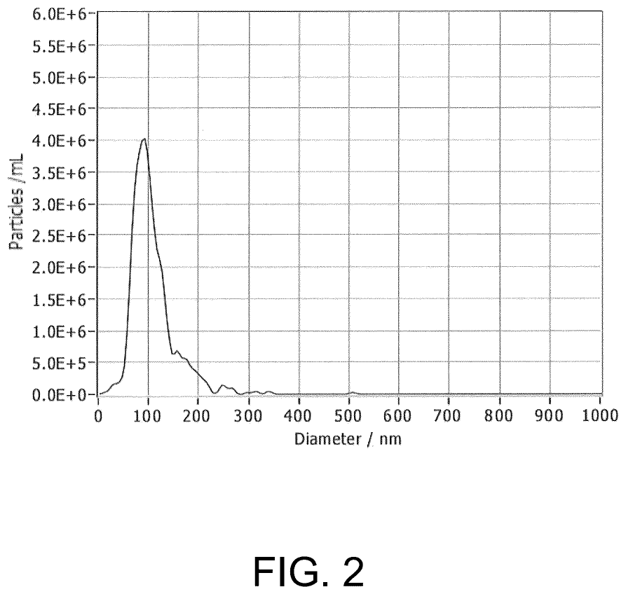

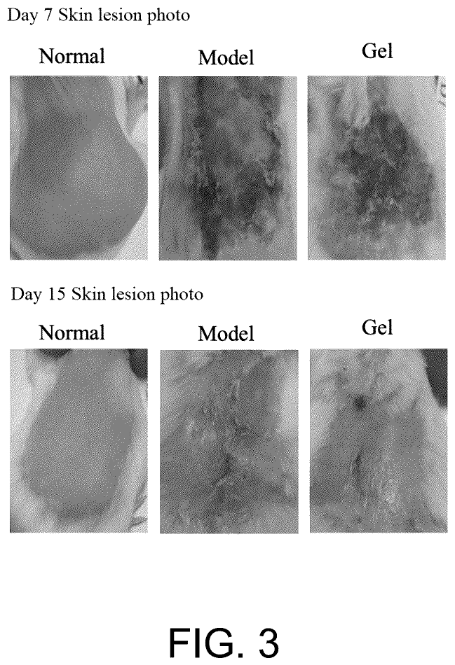

(5) Detecting the resuspension in (4) to determine an exosome content in the mesenchymal stem cells by electron microscopy, shown in FIGS. 1a and 1b, and NTA particle size analysis is shown in FIG. 2. The exosome protein content was determined by BCA as 0.28 mg/ml.

2. Mixing of the Exosomes with a Freeze-Drying Protective Agent

2 ml of exosomes prepared above were mixed with the protective agent to obtain an exosome mixture to be lyophilized. Particularly, the protective agent consists of ceramide, lysine, and glycerol. Composition of the protective agent in the exosome mixture to be lyophilized in each example and comparative example is shown in Table 1.

TABLE 1

| Item Number | ceramide (v/v %) | lysine (mM) | glycerol (v/v %) |
|---|---|---|---|
| Example 1 | 0.1 | 50 | 0.8 |
| Example 2 | 0.14 | 60 | 1.0 |
| Example 3 | 0.18 | 70 | 1.3 |
| Example 4 | 0.2 | 80 | 1.5 |
| Blank | — | — | — |
| Comparative | 0.18 | 40 | 1.3 |
| example 1 | | | |
| Comparative | 0.18 | 100 | 1.3 |
| example 2 | | | |
| Comparative | — | 70 | 1.3 |
| example 3 | | | |
| Comparative | 0.18 | 70 | — |
| example 4 | | | |

### 3. Freezing Procedures

The exosome mixture to be freeze-dried was put in liquid nitrogen and quick-frozen at −80° C. overnight, and vacuum freeze-dried by using a freeze dryer. Lyophilization conditions: cold trap temperature: −50 to −55° C., vacuum pressure 5 to 10 Pa, treatment for 24 h. After lyophilization, it was powdered and stored in 4° C. and sealed for storage.

### 4. Effect of the Freeze-Drying Protective Agent on Exosomes

### 4.1. Exosome Morphology

The lyophilized exosomes were reconstituted: a pre-cooled PBS solution was added to the lyophilized exosomes for vortex mixing, and the morphology of exosomes after reconstitution was observed.

TABLE 2

| Item Number | Exosome morphology | dispersion degree of exosomes |
|---|---|---|
| Example 1 | cup shaped vesicle structure, | No significant aggregation of exosomes |
| | relatively complete morphology, | was observed |
| | with a small amount of | |
| | membrane fusion | |
| Example 2 | cup shaped vesicle structure, | No significant aggregation of exosomes |
| | relatively complete morphology, | was observed |
| | with a small amount of | |
| | membrane fusion | |
| Example 3 | cup shaped vesicle structure, | No significant aggregation of exosomes |
| | relatively complete morphology, | was observed |
| | no obvious exosome membrane | |
| | fusion phenomenon observed | |
| Example 4 | cup shaped vesicle structure, | No significant aggregation of exosomes |
| | relatively complete morphology, | was observed |
| | with a small amount of | |
| | membrane fusion | |
| Blank | A large number of exosome | A large number of exosomes aggregated |
| | membrane structures had been | and had poor dispersion |
| | disrupted | |
| Comparative | cup shaped vesicle structure, | more exosomes gathered, with moderate |
| example 1 | relatively complete morphology, | dispersion |
| | with a small amount of | |
| | membrane fusion | |
| Comparative | cup shaped vesicle structure, | more exosomes gathered, with moderate |
| example 2 | relatively complete morphology, | dispersion |
| | moderate amount of membrane | |
| | fusion | |
| Comparative | cup shaped vesicle structure, | a small amount of exosomes aggregated, |
| example 3 | relatively complete morphology, | with good dispersion |
| | with a small amount of | |
| | membrane fusion | |
| Comparative | cup shaped vesicle structure, | more exosomes gathered, with moderate |
| example 4 | relatively complete morphology, | dispersion |
| | moderate amount of membrane | |
| | fusion | |

Table 2 lists the changes in exosome morphology in each group, As can be seen from Table 2, without the addition of a freeze-drying protective agent, there is a massive aggregation of the exosomes with a low dispersion degree, while the exosome membrane structure has broken and produced a large number of membrane fusion. It can be seen that a smaller amount of lysine in Comparative example 1, and more amount of lysine in Comparative example 2. It can be indicated from the results of Comparative examples 1 and 2 that a certain concentration of lysine plays a major role in protecting the integrity of the exosome morphology. Ceramide is absent in Comparative example 3, while ceramide was paired mainly with lysine, and assisted lysine to further reduce the exosome aggregation and membrane fusion phenomenon. Glycerol plays in preventing the generation of a lattice in the freeze-drying protective agent of the invention, and avoids the massive breakage of the exosome membrane structure. Comparative example 4 is lack of glycerol, and therefore, in contrast to the exosome aggregation, the fusion of the membrane structure of the exosome is even more obvious.

### 4.2. Exosome Biological Function

### A. Anti-Inflammatory Action

1. Test animals: male 6-week-old SPF mice.

2. Test design: 40 healthy mice with intact skin were selected and divided into 10 groups. After conventional anesthesia, the abdominal hairs were removed and the depilation area was 2 cmx 2 cm. The reconstituted exosomes from each group in Table 1 were taken and coated on the surface of the hair-removed skin. After 24 h, the skin coated with exosomes was washed with warm water, removed from the body and homogenized for the detection of immune-inflammatory factors. The detection was performed according to the existing kits, and the results are shown in Table 3.

TABLE 3

| Item Number | IL-4 (pg/mL) | IL-5 (pg/mL) | IL-6 (pg/mL) |
|---|---|---|---|
| Example 1 | 2.71 ± 0.14 | 1.82 ± 0.25 | 3.84 ± 0.31 |
| Example 2 | 2.63 ± 0.16 | 1.77 ± 0.27 | 3.62 ± 0.26 |
| Example 3 | 2.46 ± 0.12 | 1.67 ± 0.34 | 3.71 ± 0.42 |
| Example 4 | 4.28 ± 0.28 | 3.56 ± 0.17 | 4.85 ± 0.32 |
| Blank | 3.31 ± 0.26 | 3.27 ± 0.22 | 4.16 ± 0.26 |
| Comparative | 3.58 ± 0.25 | 3.11 ± 0.13 | 4.27 ± 0.17 |
| example 1 | | | |
| Comparative | 4.02 ± 0.29 | 3.46 ± 0.14 | 4.59 ± 0.26 |
| example 2 | | | |
| Comparative | 3.59 ± 0.15 | 3.13 ± 0.21 | 4.08 ± 0.32 |
| example 3 | | | |

As can be seen from Table 3, the value of the inflammatory factor in the blank control group is higher without adding the freeze-drying protective agent; the amount of lysine in the Comparative example 1 is less, and the amount of lysine in Comparative example 2 is higher; the results of Comparative example 1 and 2 show that the lysine at a certain concentration can further reduce the inflammatory response; Comparative example 3 is lack of ceramide and the value of an inflammatory factor is higher, which also shows that the addition of ceramide can reduce the inflammatory response; Comparative example 4 is lack of glycerol and the effect on the inflammatory factor is less than that of the comparative example 3.

Therefore, on the basis of protecting the integrity of the exosome morphology, the freeze-drying protective agent can further reduce the inflammatory response and the content of inflammatory factors and also play an anti-inflammatory role.

### B. Wound Repair

Wound repair ability of the reconstituted exosomes was explored, and a chemical agent was used to stimulate the mice. DNFB (dinitrofluorobenzene) is a compound commonly used in laboratory studies to induce allergic reactions in the skin, which can cause chemical damage to the skin. BALB/c mice were used, and randomly divided into DNFB positive control group (Model), DNFB+exosome group (Gel), and control group (Normal), with six mice in each group. Mice were provided with a standard diet and water and kept under routine rearing conditions in the experimental environment. DNFB was prepared with Carbomer into an ointment at the appropriate dose (0.25 to 0.5%) and applied on the back of the mice on the 1st, 4th, 7th, 10 the day from the beginning of the experiment for chemical stimulation, and administration was given to the DNFB+exosome group twice a day after the 5th day.

The results showed that on the 7th day, the skin lesion area of the Gel group was significantly less than the positive control group; on the 15th day, the Gel group was basically completely healed, while the positive control group also had a large wound area; it can be seen that the exosome after the reconstitution of Example 3 of the invention still had significant wound healing effect and showed stable biological function.

What is claimed is:

1. A freeze-drying protective agent for mesenchymal stem cell-exosomes, composed of 0.1-0.2 v/v % ceramide, lysine, and 0.8-1.5 v/v % glycerol, wherein a final concentration of lysine after mixing the freeze-drying protective agent with the mesenchymal stem cell-exosomes is 50 to 80 mM.

2. The freeze-drying protective agent according to claim 1, composed of 0.18 v/v % ceramide, lysine, and 1.3 v/v % glycerol, wherein the final concentration of lysine after mixing the freeze-drying protective agent with the mesenchymal stem cell-exosomes is 70 mM.

3. Application of the freeze-drying protective agent according to claim 1 in preparing an exosome freeze-drying preparation.

4. Application of the freeze-drying protective agent according to claim 2 in preparing an exosome freeze-drying preparation.

5. Application of the freeze-drying protective agent according to claim 1 in improvement on exosome morphology.

6. Application of the freeze-drying protective agent according to claim 2 in improvement on exosome morphology.

7. The application according to claim 5, wherein the improvement on exosome morphology refers to increasing a dispersion degree of exosomes and/or decreasing exosome membrane fusion.

8. The application according to claim 6, wherein the improvement on exosome morphology refers to increasing a dispersion degree of exosomes and/or decreasing exosome membrane fusion.

9. A mesenchymal stem cell-exosome freeze-drying preparation, containing the freeze-drying protective agent according to claim 1.

10. A mesenchymal stem cell-exosome freeze-drying preparation, containing the freeze-drying protective agent according to claim 2.
